(19) United States
(12) Patent Application Publication
(10) Pub. No.: US 2005/0165407 A1
(43) Pub. Date: Jul. 28, 2005

(54) Disk arthroplasty instrumentation and implants

(76) Inventor: Robert L. Diaz, Palm Beach Gardens, FL (US)

Correspondence Address: MCHALE & SLAVIN, P.A., 2855 PGA BLVD, PALM BEACH GARDENS, FL, 33410, US

(21) Appl. No.: 10/769,743

(22) Filed: Jan. 23, 2004

(51) Int. Cl. A61B 17/58

(52) U.S. Cl. 606/90; 606/87

(57) ABSTRACT

The present invention provides methods, surgical instrumentation and artificial implants for performing spinal disk arthroplasty and spinal facet arthroplasty. The surgical instrumentation includes a cutting guide, a distractor and a cutting block. The artificial implants for spinal disk arthroplasty can have either a fixed bearing (constrained or semi-constrained) or a mobile bearing. Additionally, the invention provides kits for performing spinal disk arthoplasty and spinal facet arthoplasty including surgical instrumentation and artificial implants.

### FIELD OF THE INVENTION

This invention relates generally to the field of surgical installation of prosthetic joints and other such devices; and particularly to methods and instrumentation for installing artificial, articulating spinal disk and spinal facet prosthetics.

### BACKGROUND OF THE INVENTION

The human spine is a flexible structure composed of bony elements termed vertebrae, which are separated and cushioned by fibro-cartilaginous sac-like structures termed intervertebral disks. The inherent elasticity of natural intervertebral disks allows for various degrees of articulation to maintain posture and a range of motion. Unfortunately, natural intervertebral disks are often damaged or destroyed by disease and/or trauma resulting in intense pain, loss of function, and possible muscular atrophy or paralysis. The conventional treatment for damaged and/or destroyed disks is surgical removal of the disk and fusion of the adjacent vertebrae. Fusion of the vertebrae removes mobility from the area and encourages degeneration of the disks above and below the fused area, thus perpetuating the original damage. Additionally, vertebral fusion often requires additional bony tissue that is obtained through autografts (bone is obtained from another part of the body) and/or allografts (bone is obtained from a donor). Allografts may result in autoimmune difficulties due to tissue rejection, thus furthering complications.

Researchers have attempted to avoid the use of vertebral fusion by creating artificial disks for insertion into the spine to replace ruptured, injured and excised natural disk material. These artificial intervertebral disks require some form of articulation or inherent flexibility to recreate the functions of the natural intervertebral disk. These artificial disks typically are rounded to fit complementarily with the vertebral surfaces and are usually composed of two articulating plate members which connect adjacent superior and inferior vertebrae. The articulating function can be produced by ball and socket joints, gel-filled enclosures, spring biased plates and plate/joint combinations. These artificial disks can be made from any bio-inert material such as plastics, ceramics, rubber, metals and combinations thereof. The artificial disk must be capable of articulation in different axes in order to provide for the changing center of rotation of adjacent vertebral surfaces, to provide for side-to-side and front-to-back translation of the vertebral surfaces relative to each other and integrate combinations of these movements in imitation of the natural intervertebral disk. The artificial disk must perform these functions without adversely affecting the spinal cord, nerves, arteries and veins near the spine.

Previously known artificial disks have had many disadvantages including migration and dislocation of the various components, polyethylene cold flow, cold-welds of metal components, ossification and degeneration of the various components resulting in the formation of debris and requiring further surgical intervention.

Intervertebral disks are not the only players functioning in the mechanics of spinal motion, the vertebrae are also composed of facet joints which provide sliding articulation, proper stiffness for avoidance of hypermobility and physiological load transmission functions. Facets damaged and/or destroyed by disease, trauma or deformation are also a source of spinal disorders. The conventional treatment is removal of the damaged/destroyed facet. However, removal may lead to hypermobility, which can lead to other disorders, thus perpetuating the problems.

There remains a need in the art for a spinal prosthetic system which can accurately re-create the natural functions of the intervertebral disks and facet joints, while simultaneously possessing the durability necessary for long-term use. In addition to providing the prosthetics, such a system should also provide the surgical instrumentation required to prepare the implantation site and to secure the prosthetic device for proper functioning.

### PRIOR ART

U.S. Pat. No. 3,867,728 discloses a prosthesis for spinal repair comprising a core member of elastic polymer having flat top and bottom surfaces.

U.S. Pat. No. 4,863,477 discloses an intervertebral disk prosthesis composed of rubber having a hollow interior that may be injected with a fluid, such as a saline solution.

U.S. Pat. No. 4,735,754 discloses methods for forming prosthetic devices having varying degrees of flexibility.

U.S. Pat. Nos. 4,309,777; 4,759,769 and 5,458,642 disclose prosthetic intervertebral disks having two plates with planar surfaces.

U.S. Pat. Nos. 4,759,766; 5,314,477; 5,556,431 and 5,562,738 disclose prosthetic intervertebral disks having two plates; each plate composed of a planar surface and a contoured surface wherein the contoured surface articulates with the contoured surface of adjacent plates.

U.S. Pat. No. 4,349,921 discloses an intervertebral disk prosthesis comprising a member with a superior surface, an inferior surface, opposing lateral surfaces and opposing anterior and posterior ends wherein each of the superior and inferior surfaces are substantially flat in the lateral-lateral direction over the entirety of surfaces and in the interior-posterior direction corresponding generally with the shape of the vertebral surface adjacent to the disk.

U.S. Pat. No. 5,401,269 discloses an intervertebral disk prosthesis comprising two articulating plates which are rotatable about a vertical axis.

U.S. Pat. No. 5,258,031 discloses an intervertebral disc comprising: 1) a first member having a first joint surface, a first anterior end and an opposing first posterior end, the anterior and posterior ends defining a transverse midline there between; 2) a second member having a second joint surface, a second anterior end and an opposing second posterior end; and 3) a ball and socket joint located between the first and second joint surfaces and between the transverse midline and the first posterior end. The ball and socket joints permits relative rotation of the first and second member about a first axis parallel to the transverse midline and about a second axis perpendicular to the first axis.

U.S. Pat. No. 5,425,773 discloses an intervertebral disc comprising: 1) a first member having a first joint surface, a first anterior end and an opposing first posterior end, the anterior and posterior ends defining a transverse midline there between; 2) a second member having a second joint surface, a second anterior end and an opposing second posterior end; and 3) a ball and socket joint between the first and second joint surfaces and between the transverse midline and the first posterior end. The ball and socket joints permits relative rotation of the first and second member about a first axis parallel to the transverse midline and about a second axis perpendicular to the first axis. Additionally, at least one of the first and second joint surfaces is inclined away from the ball and socket joint entirely around the joint, and the other one of the first and second joint surfaces lies along a plane substantially parallel to both the first and second axes.

U.S. Pat. No. 5,676,701 discloses a low wear artificial spinal disc comprising: 1) a first component including a recess having a contoured surface with a 360° circumference; and 2) a second component including a projection having a contoured surface with a 360° circumference. The contoured surfaces permit unrestricted rotational motion and a flexion/extension bending motion between the components relative to a standing patient's spinal axis. The flexion/extension angle is between about 20-30°.

U.S. Pat. No. 5,514,180 discloses intervertebral prosthetic devices having fixed shapes for accommodating the defined surface contours of the vertebral endplates. The invention defines five morphological types of surfaces comprising a set of surfaces capable of accommodating the anatomy of most vertebral endplates. A method of digitizing the surface of a vertebral body to determine a specific shape of a vertebral endplate is also disclosed. Furthermore this invention also relates to such prosthetic devices incorporating osteoinductive material such as bone growth factors.

U.S. Pat. No. 5,545,229 discloses an intervertebral disk spacer comprising a central core of soft elastomer approximating the size and shape of the nucleus pulposus, an outer ring of stiffer elastomeric material approximating the size and shape of the annulus fibrosis, and endplates of stiff material incorporating a mechanism for attachment to the adjacent bony vertebral bodies.

U.S. Pat. Nos. 5,376,323; 5,855,606 and 5,700,288 disclose hollow prostheses comprising room temperature vulcanizable silicone.

U.S. Pat. No. 5,314,478 discloses a prosthesis that can be used as a replacement for intervertebral disk. This prosthesis is a composite of polyvinyl alcohol hydrogel and a ceramic or metallic porous body.

U.S. Pat. No. 5,824,093 discloses a prosthetic spinal disk comprising a jacket surrounding a hydrogel core that hydrates to a pre-determined volume.

U.S. Pat. No. 5,702,454 discloses a prosthetic implant for replacing a spinal disk. Support members are inserted into a cavity in the core of the disk until the cavity is filled.

Various other intervertebral disk prosthetics are described in U.S. Pat. No. 5,071,437; 6,113,637; 6,001,130; 5,527,312; 6,039,763; 6,146,421; 5,123,926; 5,306,307;6,283,998; 6,146,419 and 5,824,094.

Still other artificial disk prosthetics and methods for disk replacement are known in the art.

U.S. Pat. No. Re-Issue 36,758 discloses an artificial facet joint wherein the inferior facet or the superior facet or both are covered by a cap.

U.S. Pat. No. 6,132,464 discloses a spinal facet joint prosthesis that is supported on the posterior arch (lamina). The support structure has inferior and/or superior blades extending from it which replace cartilage at the facet joint.

WO 98/48717 A1 discloses a technique for surgical removal and replacement of the spinal facets in a manner that immobilizes the joint.

U.S. Pat. No. 6,565,605 and 6,419,703 also disclose methods and prosthetics for spinal facet replacement.

### SUMMARY OF THE INVENTION

The present invention provides a spinal prosthetic system which can accurately re-create the natural functions of the intervertebral disks and facet joints, while simultaneously possessing the durability necessary for long-term use. In addition to providing the prosthetics, this system also provides the surgical instrumentation required to prepare the implantation site and to secure the prosthetic device for proper functioning. The instrumentation provided by the instant invention enables the preparation of the inferior and superior vertebral surfaces through either an anterior or lateral approach. The surfaces of the vertebrae are surgically modified as necessary for maximum contact with the implant and to prevent trauma to the spinal cord and blood supply. The cutting guide (sizing instrument) prepares and sizes the vertebral incisions and has a movable handle which can be placed for lateral or anterior insertion into the disk space. The distractor is attached to each of the adjacent vertebrae and functions to separate and stabilize the vertebrae until the prosthesis is installed. The cutting block is inserted into the disk space to guide the cutting instruments and to protect body tissues (for example, nerves and blood vessels). The cutting block may also have an attached retractor to provide additional protection.

The instant invention provides artificial disk implants with three types of bearings; fixed, semi-constrained bearing, a fixed, constrained bearing and a mobile bearing. The components of the artificial implants are capable of varying degrees of motion due to the type of bearing and are attached to the inferior and superior surfaces of adjacent vertebrae wherein a natural disk has been removed.

Additionally, the instant invention provides artificial implants for the repair or replacement of the vertebral facets.

The disk implants and the vertebral facet implants can be installed either separately or together and can be installed in any order; for example, the disk implants can be installed either before or after the vertebral facet implants.

Accordingly, it is an objective of the instant invention to provide a method of installing an articulating, intervertebral disk prosthetic device wherein the device has a fixed, constrained bearing, a fixed, semi-constrained bearing or a mobile bearing.

It is another objective of the instant invention to provide a method of installing prosthetic devices for repair of vertebral facets.

It is still another objective of the instant invention to provide a method of installing both an intervertebral disk prosthetic device wherein the device has a fixed, constrained bearing, a fixed, semi-constrained bearing or a mobile bearing and a prosthetic device for repair of vertebral facets within the same surgical procedure.

It is yet another objective of the instant invention to provide a method for preparing a vertebral site for implantation of an intervertebral disk prosthetic device and/or prosthetic devices for the repair of vertebral facets.

It is an objective of the instant invention to provide a cutting guide to prepare a vertebral incision for lateral or anterior insertion of a prosthetic device into a disk space.

It is another objective of the instant invention to provide a distractor attached to each of the adjacent vertebrae which functions to separate and stabilize the vertebrae until the prosthesis is installed.

It is still another objective of the instant invention to provide a cutting block for guiding cutting instruments and protecting body tissues.

It is another objective of this invention to provide an articulated, intervertebral disk implant with relatively movable components attached to the inferior and surperior surfaces of adjacent vertebrae.

It is a further objective of the instant invention to provide an articulated, intervertebral disk with a fixed bearing that is either constrained or semi-constrained. The fixed bearings snap into place by attachment to the inferior body of the implant.

It is still another objective of the instant invention to provide an implant with a mobile bearing.

It is a still further objective of the instant invention to provide implants for the superior and inferior vertebral facets.

It is yet another objective of the instant invention to provide surgical kits for disk arthroplasty and/or vertebral facet arthroplasty containing artificial prosthetic devices and the surgical instrumentation required to install such artificial prosthetic devices.

Other objects and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

FIG. 1 is top plan view of the superior surface of the inferior vertebra and the cutting guide (sizing instrument) of this invention;

FIG. 2 is a lateral view of the inferior and superior vertebrae;

FIG. 3 is an anterior view of the inferior and superior vertebrae;

FIG. 4 is a perspective view of the distractor of this invention;

FIG. 5 is a perspective view of the cutting block of this invention;

FIG. 6 is a side view, partially in section, of an fixed bearing implant of this invention;

FIG. 7 is a plan view of the implant of FIG. 6;

FIG. 8 is a side view of another embodiment of the implant of this invention with a semi-constrained bearing;

FIG. 9 is a cross section of FIG. 8 along line 9 -9;

FIG. 10 is a side view of another embodiment of the implant of this invention with a constrained bearing;

FIG. 11 is a side view of the implant with a mobile bearing;

FIG. 12 is a top planar view of the inferior and superior vertebrae with the posterior facets; and

FIG. 13 is an anterior view of the implants for repair of the vertebral facets.

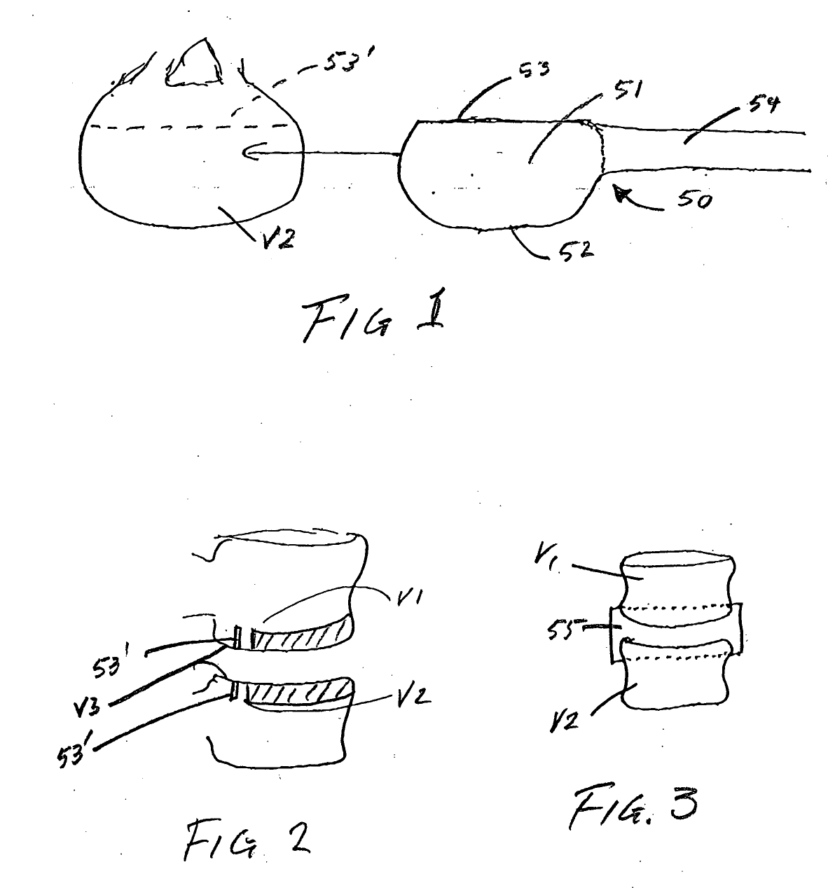

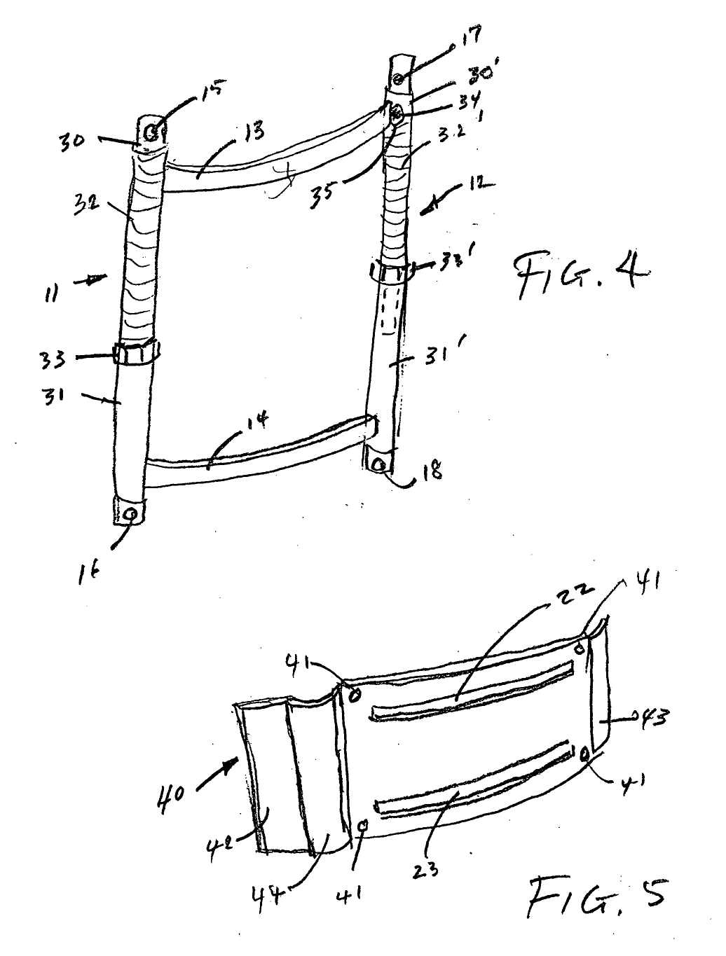

### DEFINITIONS AND ABBREVIATIONS

The following list defines terms, phrases and abbreviations used throughout the instant specification. Although the terms, phrases and abbreviations are listed in the singular tense the definitions are intended to encompass all grammatical forms.

As used herein, the term “natural intervertebral disk” refers to a biological disk present in the body as opposed to a “man-made” artificial disk.

The terms “prosthesis”, “implant” and “artificial disk” are used interchangeably herein.

The terms “cutting guide”, “sizing instrument” and “guide” are used interchangeably herein.

The terms “spinal disk” and “intervertebral disk” are used interchangeably herein.

The terms “vertebral facet” and “spinal facet” are used interchangeably herein.

### DETAILED DESCRIPTION

At the beginning of the surgical procedure, access to the spinal column is gained by either a lateral or an anterior approach. The condition of the disk may determine how the procedure will continue. For purposes of illustration of the invention, in FIG. 1, a guide 50 (also referred to as a sizing instrument or cutting guide) is inserted into the disk space. The guide 50 has a thin planar forward end 51 that has a curved edge 52 shaped to approximate the shape of the anterior portion of the vertebral surfaces of the adjacent vertebrae. Opposite the curved edge 52, the forward end has a straight edge 53. A handle 54 is formed on one side of the forward end 51 extending outwardly from the straight edge. The location of the handle determines whether the approach will be lateral or anterior. The forward end is inserted into the disk space with the curved edge contiguous with the anterior periphery of the adjacent vertebrae and the straight edge 53 traversing the superior and inferior surfaces of the vertebrae. In order to determine vertebral size, the guide is palpated around the periphery of the disk space. An incision 53′ is made in both vertebrae along the straight edge of the guide. This incision may be made by a reciprocating saw blade perpendicular to the straight edge. The reciprocating blade moves backward and forward along the straight edge and may incise both vertebrae at the same time. A thin plate 55 may be inserted in each incision and serves to prevent any penetration toward the spinal cord and blood supply. If such a plate 55 is inserted, it remains in place until after the prosthesis is implanted.

The distractor 10, shown in FIG. 4, is used to translate and stabilize the adjacent vertebrae V1 and V2. The distractor has a frame formed by a left vertical rail 11 and a right vertical rail 12. The rails 11, 12 have an upper leg 30, 30′ and a lower leg 31, 31′ which telescope together using, for example, a worm gear. The upper leg has external screw threads 32, 32′. The rails are connected near their respective ends by a top cross member 13 and a bottom cross member 14. The cross members are slightly curved to maintain a close relationship with the circumference of the vertebrae. The cross member 13 may be connected to the upper leg by pins 34 inserted into larger holes 35 to allow uneven or non-parallel movement of the upper legs and cross member, without binding. Each leg has a pair of apertures15, 16 and 17, 18, respectively, near the ends for the insertion of pins or screws into the adjacent vertebrae. The distractor 10 is mounted on the adjacent vertebrae spanning the disk space in the closed position and the distractor is fastened to the vertebrae through the apertures 15, 16, 17 and 18.

The distractor is expanded to simulate the original disk space by telescopically moving the upper and lower legs. The cross member 13 moves away from cross member 14 in response to a threaded nut 33, 33′. As shown, the threads of the nut 33, 33′ engage the external threads of the upper leg 30, 30′ so that turning the nut translates the upper leg. Because there is some angular clearance in the telescoping components, each lag may be moved individually to some extent or both may be moved together. The mechanism for expanding the cross members may also be hydraulic, air pressure, scissor jack, worm gear or other device.

Once the adjacent vertebrae are positioned to approximate the natural location, a cutting block 40 may be placed on the rails 11, 12, depending on the condition of the inferior and superior surfaces of the vertebrae. In some cases, the surfaces do not require major excision and the cutting block may not be necessary. If desired, the intervertebral disk can be removed, the end plates curetted and the prosthesis inserted without bone cuts. The cutting block has a cutting slots 22 and 23. The cutting slots captures a saw blade or other cutter used to prepare the inferior surface of the higher vertebrae and the superior surface of the lower vertebrae. The posterior portion of each vertebrae is not excised and provides a ridge of bone V3 preventing rearward migration of the implant.

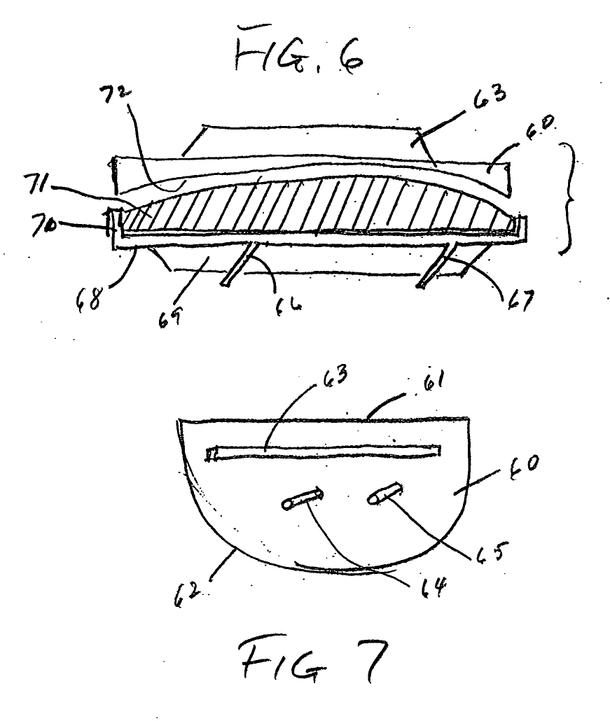

The cutting block 40, shown in FIG. 5, may be attached to the rails 11, 12 by resilient clips, pins or screws (not shown) through flanges 43, 44 or the block may have screw holes 41 on each corner through which pins may be inserted into the adjacent vertebrae. In one embodiment, the cutting block has a retractor 42 along one side to move critical tissue, such as arteries, veins, nerves, out of the surgical field. The retractor 42 may be permanently affixed to the block or removable.

Once the surfaces of the vertebrae are prepared, the cutting block is removed from the vertebrae. The site is now ready for the disk implant to be inserted into the intervertebral space. The distractor remains in place until after the prosthetic device is implanted.

Several embodiments of the implant are illustrated but all the implants have a superior body to be attached to the superior vertebral surface, an inferior body to be attached to the inferior surface and a bearing captured between the superior and inferior bodies. The superior and inferior bodies have shaped opposing seats complementary to the surface of the bearing. In this manner, the bearing allows front to back, side to side, rotational and combinations thereof, movement along the spinal column. The components (inferior body, superior body, bearings) of the implant are composed of bio-inert materials. Illustrative, albeit non-limiting, examples of such bio-inert materials are surgical stainless steel and other metals, ceramics, polymers, polyethylene, hedrecel and various combinations thereof.

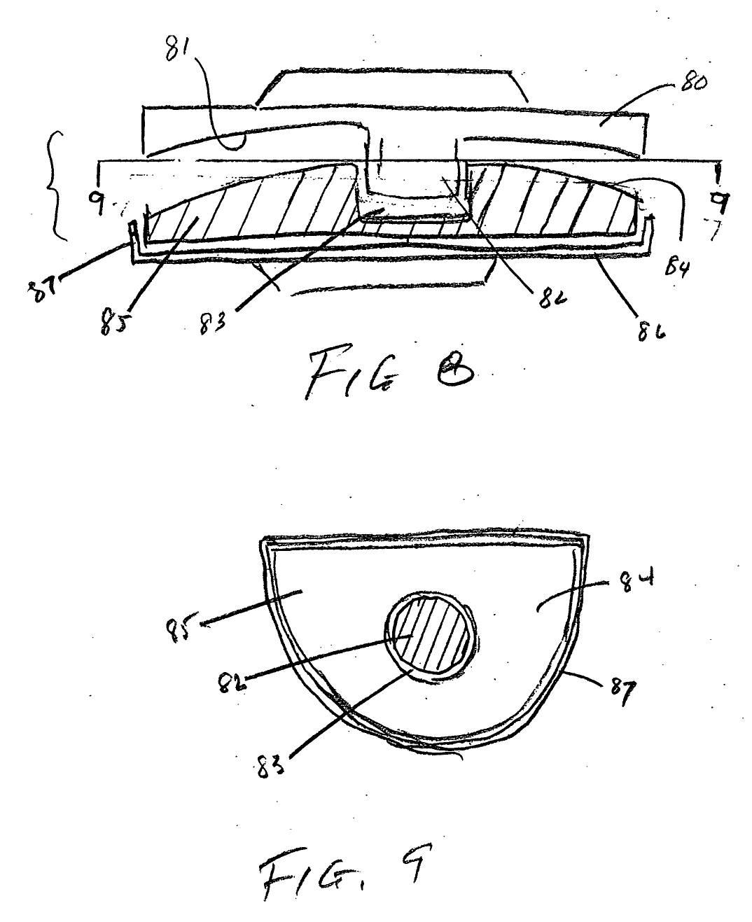

The implant shown in FIG. 6 and FIG. 7 has an superior body 60 and an inferior body 68 which may be made of surgical stainless steel or other bio-inert materials such as ceramics, polymers or other metals. The surfaces are shaped to fit the excised area of the superior and inferior surfaces of the vertebrae.

The superior body 60 has a straight edge 61 and a curved front edge 62. A keel 63 is formed tranversely on the upper surface for insertion in the inferior surface of the vertebrae, either in the transverse incision of by impaction. Pegs 64, 65 are also formed on the upper surface similar to the pegs 66, 67 on the inferior body 68. According to the surgical approach to the spine, all the pegs may be angled to slide into pilot holes in the vertebrae. As shown, the pegs are angled for a lateral approach. The lower surface is formed concave to provide a seat 72 for the bearing 71.

The inferior body 68 also has a keel 69 inserted into the inferior vertebrae. Keels are helpful to prevent posterior migration of the prosthesis. The inferior body 68 has a peripheral wall 70 about the perimeter to retain the bearing 71.

The bearing 71 may be made of any bio-inert material that will withstand the friction and anatomical forces generated in the movement of the spine, for example polyethylene, or other polymers, or ceramics, or metals, or polymers laminated to metals. The bearing has a convex or conical upper surface complementary to the concave surface 72 of the superior body 60 to allow for universal motion in the spinal column. The bearing may be somewhat smaller than the dimensions of the peripheral wall 70 to permit greater range of motion. When using a fixed bearing the radius of articulation determines the range of motion. This fixed bearing snaps into place and is firmly held, thus avoiding complications with dislocation of the bearing.

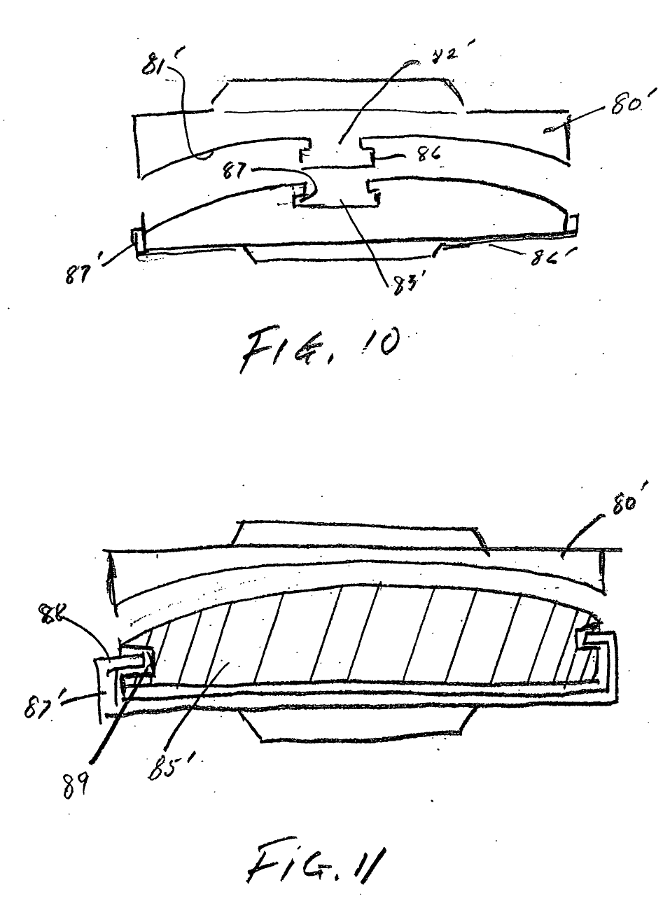

Another embodiment of the spinal disk implant is shown in FIGS. 8 and 9. The superior body 80 is formed similar to the superior body 60 and is affixed to the spine in the same fashion. The inferior body 86 is formed in a similar fashion to the inferior body 68 with a peripheral wall 87. The concave lower surface 81 has a cylindrical spindle 82 depending downwardly. The spindle 82 is received in a cylindrical depression 83 formed in the complementary upper surface 84 of the bearing 85, as shown in FIG. 9. The diameter of the depression 83 is somewhat larger than the diameter of the spindle 82 to allow a lesser degree of movement.

Another implant embodiment similar to the embodiment of FIG. 8 and 9 is shown in FIG. 10. The spindle 82′ has a circular enlargement 86 on the end. The bearing depression 83′ has a complementary circular enlargement 87 into which the spindle is snap fit. This arrangement provides a more secure articulation with a lesser degree of movement.

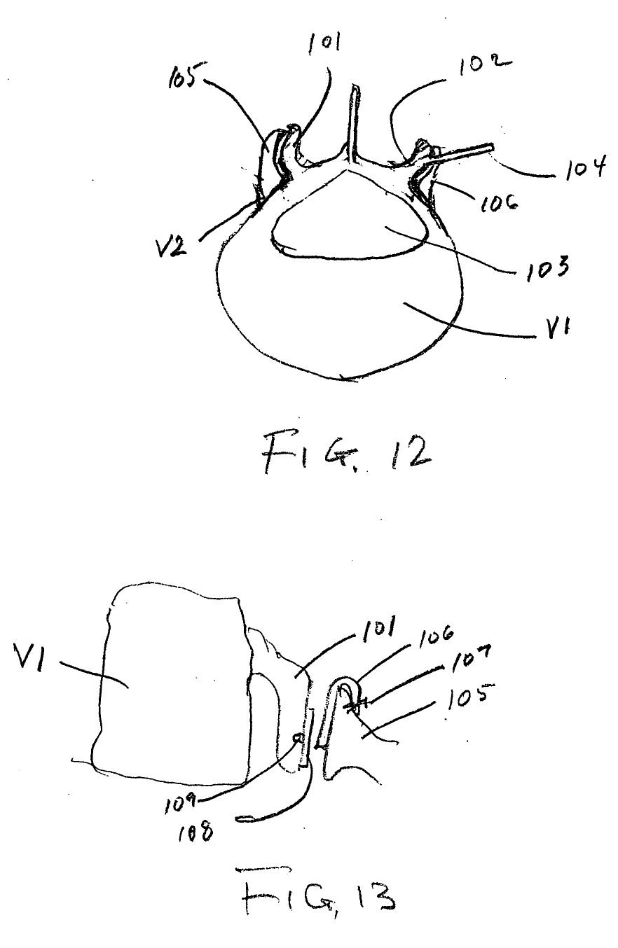

The embodiment of FIG. 11 has a smaller peripheral ring 88 formed on the peripheral wall 87′. The bearing 85′ has a circumferential groove 89. The ring 88 is snap fit into the groove 89 to secure the bearing in the inferior body. The mobile bearing 85′ articulates with a polished surface on both it's superior and inferior surfaces, and it's motion is limited by the size of the restraining ring 88 as compared with the indentation in the bearing 85′. A portion of the circumference of the ring 88 is removed to allow insertion and is then fixed firmly in place using screws or another such device to hold it in place.

The implants can be fixated to the bone using a variety of techniques. Illustrative, albeit non-limiting examples, are angled or straight metal pegs with cement fixation, poly impregnated into hedrecel base using hedrecel pegs, porous coated base with angled or straight porous coated pegs and angled metal spikes and cement fixation. Keeling at the posterior end of a metal prosthesis will help fixation and prevent posterior migration of the prosthesis.

In conjunction with the disk implant described above, or independently therefrom, the facets of the adjacent vertebrae may receive implants. As shown in FIG. 12, the superior and the inferior vertebrae have posterior wing-like projections or facets which are in contact with each other. It may be necessary to surgically intervene to repair these joints. In FIG. 12, the superior vertebra V1 has facets 101 and 102 the spinal canal 103 and a lateral process 104. The other lateral process is not shown for clarity. The inferior vertebra V2 is directly below V1 with only the inferior facets 105, 106 visible. Along the spinal column and as shown in FIG. 12 and FIG. 13, the inferior facet 105 is outside the superior facet 101. As shown in an anterior view in FIG. 13, the inferior facet 105 has an implant shaped as a cap 106 fixed to the facet by a pin or screw 109. The superior facet has a button shaped implant 108 attached to the facet by a peg or screw 109. The first implant for the superior facet can be composed of a polished metal and the second implant for the inferior facet can be composed of polyethylene and attached with a peg or polyethylene backed with a metal or hedrecel using a keel. The polyethylene can be fixated using methyl methacrylate and the metal or hedrecel backed with a keel can be cemented into place.

Upon completion of the surgical procedures, the disk space is closed. The opening is covered and sealed with mesh attached by anchors superior and inferior to the vertebral bodies.

All of these surgical components, described above, may be included in a surgical pack or kit for convenience for use in disk and/or facet arthroplasty.

In conclusion, as shown by all of the above description, the present invention provides a spinal prosthetic system, including prosthetic devices and instrumentation for installation of such devices, which can accurately re-create the natural functions of the intervertebral disks and facet joints, while simultaneously possessing the durability necessary for long-term use.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is well adapted to carry out the objectives and obtain the ends and advantages mentioned, as well as those inherent therein. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A cutting guide for preparing vertebral incisions, comprising:
a) a planar forward end having a curved edge and a straight edge, wherein said curved edge is shaped to approximate the shape of the anterior portion of the vertebral surfaces of adjacent vertebrae and said straight edge is shaped to guide a surgical blade making an incision; and
b) a handle extending outward from said straight edge of said planar forward end.

2. The cutting guide in accordance with claim 1 wherein said handle is placed for lateral insertion into a disk space.

3. The cutting guide in accordance with claim 1 wherein said handle is placed for anterior insertion into a disk space.

4. A distractor for separating and stabilizing adjacent vertebrae comprising:
a frame comprising;
a) a right vertical rail and a left vertical rail wherein said right vertical rail and said left vertical rail each comprise an upper leg having external screw threads and a lower leg without external screw threads and wherein each of said upper legs and each of said lower legs have an aperture near the end portion;
b) a top cross member connecting said upper legs; and
c) a bottom cross member connecting said lower legs.

5. A cutting block for preparing vertebral surfaces for prosthesis implant comprising:
a) a plate having an upper horizontal cutting slot and a lower horizontal cutting slot;
b) a flange on a right side of said plate; and
c) a flange on a left side of said plate.

6. The cutting block in accordance with claim 5 wherein said plate of a) further comprises a hole at each corner.

7. The cutting block in accordance with claim 5 further comprising a retractor affixed along the side of either said flange of b) or said flange of c).

8. The cutting block in accordance with claim 6 further comprising a retractor affixed along the side of either said flange of b) or said flange of c).

9. An artificial implant for spinal disk replacement comprising;
a) a superior body for attachment to a superior vertebral surface;
b) an inferior body for attachment to an inferior vertebral surface; and
c) a fixed, constrained bearing between said superior body and said inferior body wherein a surface of said fixed, constrained bearing is complementary to a surface of said superior body and a surface of said inferior body.

10. The artificial implant in accordance with claim 9 wherein said superior body, said inferior body and said fixed, constrained bearing are composed of a bio-inert material selected from the group consisting of surgical stainless steel, ceramics, polymers, metals, polyethylene and combinations thereof.

11. An artificial implant for spinal disk replacement comprising;
a) a superior body for attachment to a superior vertebral surface;
b) an inferior body for attachment to an inferior vertebral surface; and
c) a fixed, semi-constrained bearing between said superior body and said inferior body wherein a surface of said fixed, semi-constrained bearing is complementary to a surface of said superior body and a surface of said inferior body.

12. The artificial implant in accordance with claim 11 wherein said superior body, said inferior body and said fixed, semi-constrained bearing are composed of a bio-inert material selected from the group consisting of surgical stainless steel, ceramics, polymers, metals, polyethylene and combinations thereof.

13. An artificial implant for spinal disk replacement comprising;
a) a superior body for attachment to a superior vertebral surface;
b) an inferior body for attachment to an inferior vertebral surface; and
c) a mobile bearing between said superior body and said inferior body wherein a surface of said mobile bearing is complementary to a surface of said superior body and a surface of said inferior body.

14. The artificial implant in accordance with claim 13 wherein said superior body, said inferior body and said mobile bearing are composed of a bio-inert material selected from the group consisting of surgical stainless steel, ceramics, polymers, metals, polyethylene and combinations thereof.

15. An artificial implant system for the repair of vertebral facets comprising:
a) a first implant shaped as a cap for fitting over and attaching to an inferior vertebral facet; and
b) a second implant shaped as a button for attachment to a superior vertebral facet.

16. The artificial implant system in accordance with claim 15 wherein said first implant is composed of polished metal and said second implant is composed of polyethylene or polyethylene backed by metal.

17. A surgical kit for disk arthroplasty comprising instruments and an artificial implant wherein said instruments are a sizing instrument, a distractor and a cutting block and said artificial implant has a fixed, constrained bearing.

18. A surgical kit for disk arthroplasty comprising instruments and an artificial implant wherein said instruments are a sizing instrument, a distractor and a cutting block and said artificial implant has a fixed, semi-constrained bearing.

19. A surgical kit for disk arthroplasty comprising instruments and an artificial implant wherein said instruments are a sizing instrument, a distractor and a cutting block and said artificial implant has a mobile bearing.

20. A surgical kit for vertebral facet arthroplasty comprising instruments, a first artificial implant and a second artificial implant wherein said instruments are a sizing instrument, a distractor and a cutting block and said first artificial implant is shaped like a cap for fitting over and attaching to an inferior vertebral facet and said second artificial implant is shaped as a button for attaching to a superior vertebral facet.

21. A method for preparing a vertebral site for receiving an artificial implant comprising the steps of:
a) measuring the vertebral space with a sizing instrument;
b) making a first incision in a superior vertebrae along a straight edge of said sizing instrument;
c) making a second incision in an inferior vertebrae along a straight edge of said sizing instrument;
d) stabilizing said superior vertebrae and said inferior vertebrae by mounting a distractor between said superior vertebrae and said inferior vertebrae;
e) expanding said distractor to approximate a natural disk space between said superior vertebrae and said inferior vertebrae;
f) inserting a cutting block into said distractor and preparing the inferior surface of said superior vertebrae and the superior surface of said inferior vertebrae by excising damaged disk material; and
g) removing said cutting block and said distractor whereby said vertebral site is prepared to receive said artificial implant.

22. The method in accordance with claim 21 further comprising a step of inserting an artificial implant into a prepared vertebral site wherein said artificial implant has a fixed bearing or a mobile bearing.

23. The method in accordance with claim 22 wherein said fixed bearing is constrained or semi-constrained.
